(19) United States
(12) Patent Application Publication
(10) Pub. No.: US 2015/0105463 A1
(43) Pub. Date: Apr. 16, 2015

(54) Sodium Pyruvate Oral Rehydration Salt Composition for Treating Hypovolemia or Hyponatration Associated with Hypohydration

(71) Applicant: Fangqiang Zhou, (US)

(72) Inventors: Fangqiang Zhou, Shanghai (CN); Sen Hu, Beijing (CN)

(21) Appl. No.: 14/385,673

(22) PCT Filed: Mar. 20, 2013
(86) PCT No.: PCT/CN2013/072929

§ 371 Date: Sep. 16, 2014

(30) Foreign Application Priority Data
Mar. 20, 2012 (CN) 201210074342.9

(51) Int. Cl. A61K 31/19

(52) U.S. Cl. CPC A61K 31/19
USPC 514/557

(57) ABSTRACT

Disclosed in the present invention is a sodium pyruvate oral rehydration salt composition for treating hypovolemia or hyponatration associated with hypohydration, said composition containing the following components: (i) 2.0-6.0 parts by weight of sodium pyruvate; (ii) 1.5-17.0 parts by weight of sodium chloride; (iii) 0-2.0 parts by weight of potassium chloride; and (iv) 10.0-50.0 parts by weight of glucose anhydrous or other carbohydrates. The weight of components (i)+(ii)+(iii)+(iv) constitutes 50-100% of the total weight of the composition.

### FIELD OF THE INVENTION

The invention relates to oral pharmaceutical products of sodium pyruvate-containing oral rehydration salt (Pyruvate-enriched Oral Rehydration Salt, Pyr-ORS) composition, particularly to an ORS composition used in the treatment of hypovolemia in the blood circulation system or a state of dehydration with salt loss, such as shock and diarrhea.

### BACKGROUND ARTS

In the treatment of hypovolemic shock induced by severe hemorrhage (blood loss), trauma and dehydration, especially the large area burn (scald) injury, early and aggressive intravenous fluid infusion is a key medical intervention. However, in war, serious natural disasters, epidemics or incidents (such as forest fires, terrorist attacks) scenes, batch wounded with shock often appears in a short time and medical conditions is limited due to environmental and other difficulties, the implementation of conventional intravenous fluid management (such as transfusion and infusion) is difficult or delayed, leading to battle casualties, injuries or death increased with a high incidence of complications⁽¹⁾. Animal experiments and clinical trials have shown that the oral or enteral replenish of oral rehydration salt solutions (Oral Rehydration Solutions, ORS) is an effective means of anti-shock in burns. The ORS is absorbed into the bloodstream through the gastrointestinal tract, the vital signs of the wounded can be quickly stabilized to win time for the subsequent treatments, or significantly reduce the amount of intravenous fluids. The ORS would play a special role with a high value of applications in the war, sudden accidents, natural disasters and other on-site treatment processes⁽²⁾.

Currently, acute diarrhea is still the world's second major cause of death in children diseases, each year there are around 2 million children died from diarrhea, mainly due to dehydration and salt loss. For over 30 years, the World Health Organization (WHO) has promoted the use of oral rehydration salt to save millions lives of diarrhea patients in developing countries, greatly reducing the mortality rate of diarrhea in children worldwide. The discovery of intestinal epithelial ‘Sodium-Glucose Cotransporter’ half a century ago and the development and promotion of oral rehydration salt based on the findings of the transporter were hailed by the authoritative medical journal, Lancet, as the most important medical advance in the 20th century⁽³⁾. The latest hypotonic formula to further improve the clinical effect in the treatment of diarrhea in children has been listed as one of national essential drugs in many countries, including China, over years. It will effectively promote the two-thirds reduction of global child mortality by 2015 on the basis of 1990, proposed by WHO as the UN Millennium Development Goals⁽⁴⁾.

For decades, several experimental studies and clinical applications with the standard WHO formula or modified ORS (total osmolarity of 331 mOsm/L or 270 mOsm/L) have been reported in fluid resuscitation of hypovolemic shock due to bleeding, burns and adult acute diarrhea. They could effectively replace intravenous fluids in the expansion of blood volume to maintain blood pressure and prolong life⁽⁵⁻⁹⁾. The ORS was developed at the beginning of the 1970s under the WHO guideline and recommended as the first-line product in the treatment of cholera and acute diarrhea in children worldwide. In the past 30 years, the WHO Recipe of oral rehydration salt (WHO-ORS) has been continuously improved with three major formulae:

I. The early standard formula: a liter of water contains sodium chloride 3.5 g, potassium chloride 1.5 g, sodium bicarbonate 2.5 g and 20 g of anhydrous glucose with a total osmolarity of 331 mOsm/L;
The improved formula in the 1990s: a liter of water contains sodium citrate 2.9 g replaced sodium bicarbonate in the standard ORS with a total osmolarity of 311 mOsm/L;
The hypotonic formula issued in 2006 jointly by the WHO and UNICEF: a liter of water contains sodium chloride 2.5 g, potassium chloride 1.5 g, sodium citrate 2.9 g and anhydrous glucose 13.5 g with a total osmolarity of 245 mOsm/L.

Among them, equimolar sodium citrate instead of sodium bicarbonate in formulae is to overcome the shortcomings of bicarbonate: instability of aqueous solution and gastrointestinal bloating, improve the oral taste and facilitate the long-term preservation (a long shelf life) of salts for convenient clinical applications. However, their effects are similar in preventing acid-base disturbances in diarrhea and cholera. Recent findings show that citrate, compared with bicarbonate, in ORS may in certain degree improve the intestinal mucosal blood flow and increase intestinal water and salt absorption with the reduction of fluid loss and oral fluid requirement, but, the similar duration of diarrhea. The recent hypotonic formulation, namely ORS III, Chinese products as oral rehydration salt III (Bo Ye), may be more suitable for non-cholera diarrhea, enabling early recovery of intestinal absorption of nutrients.

The key factor that influences the ORS effect on hypovolemic shock and inflammatory bowel disease is intestinal ischemia and inflammation, which induce local hypoxia in gastrointestinal mucosal cells and impair energy metabolism and intestinal barrier function, resulting in the inhibition of gastrointestinal emptying and absorption of water, salt and sugar, so that the gastrointestinal tract may be difficult to tolerate oral rehydration salts: an inappropriate ORS may cause abdominal distention, vomiting, and diarrhea, and even lead to serious consequences. When they damage barrier function, hypoxia and inflammatory in bowel can bring intestinal bacteria and endotoxin by the intestinal lymphatic or portal venous system into the blood circulation and even induce sepsis and multiple organ dysfunction⁽¹⁰⁾.

While existing WHO-ORS has been constantly improved, so far its components, per se, do not play directly and obviously therapeutic effects on bowel function in diarrheal diseases, but rather the alterations of components focused on improving the concentration and osmotic pressure, relieving gastrointestinal symptoms; or adding the auxiliary components to favor the gastric emptying of ORS and the enhancement of intestinal absorption and antimicrobial properties, etc.⁽¹¹⁻¹⁴⁾. Accordingly, an urgent need in the art is required to further improve the composition of WHO-ORS to achieve the direct preservation of intestinal cell metabolism and function against hypoxia and/or inflammatory stress and further enhance the resuscitation effect of oral rehydration on hypovolumic shock.

### SUMMARY OF THE INVENTION

The present invention aims to provide an ORS composition that contains sodium pyruvate (Pyruvate) used for the treatment of circulatory hypovolemia or dehydration with salt loss, such as shock and diarrhea.

In a first aspect of the present invention, there is provided a method for the treatment of circulatory hypovolemia or dehydration with an ORS composition contained pyruvate sodium salt, said composition comprising the following components:

(i) 2.0-6.0 parts by weight of sodium pyruvate;
1.5-17.0 parts by weight of sodium chloride;
0-2.0 parts by weight of potassium chloride or corresponding equivalents of potassium citrate; and
10.0-50.0 parts by weight of anhydrous glucose or other carbohydrates;
And the weight of the total component (i)+(\ddot{υ})+(iii)+(iv) is 50 to 100% of the total weight of the composition.

In another preferred embodiment, the composition comprising:

3.0-4.0 parts by weight of sodium pyruvate;
2.0-5.0 parts by weight of sodium chloride;
1.0-1.8 parts by weight of potassium chloride or corresponding equivalents of potassium citrate; and
13.5-20.0 parts by weight of anhydrous glucose or other carbohydrates.

In another preferred embodiment, the composition is an oral solution, said solution comprising in 1000 ml:

2.0-6.0 g of sodium pyruvate;
1.5-17.0 g of sodium chloride;
0-2.0 g of potassium chloride or corresponding equivalents of potassium citrate; and
10.0-50.0 g of anhydrous glucose or other carbohydrates

In another preferred embodiment, the solution contained in 1000 ml:

3.0-4.0 g of sodium pyruvate;
2.0-5.0 g of sodium chloride;
1.0-1.8 g of potassium chloride or corresponding equivalents of potassium citrate; and
13.5-20.0 g of anhydrous glucose or other carbohydrates.

In another preferred embodiment, the osmotic pressure of solution is 300-1000 mOsm/L, and 1000 ml of the solution containing equal to or greater than 3.5 g of sodium chloride and containing equal to or greater than 20.0 g of anhydrous glucose or other carbohydrates.

In another preferred embodiment, the osmotic pressure of solution is 120-280 mOsm/L, and 1000 ml of the solution containing less than 3.5 g of sodium chloride and containing less than 20.0 g of anhydrous glucose or other carbohydrates.

In another preferred embodiment, the composition further contains zinc, magnesium, selenium and calcium salts, phosphates, antioxidants, gastrointestinal and vascular active ingredients, immune nutrients, Chinese medicine, and/or flavoring agents. Said antioxidants are selected from the following combinations of one or more of vitamin C, vitamin E, N-Acetylcysteine and Pentoxifylline; described gastrointestinal motility and vascular active ingredients are selected from the following combinations of one or more of Carbachol, Mosapride, Anisodamine and Dopa Phenol Butylamine (Dobutamine); said immune nutrients are selected from the following combinations of one or more of L-isoleucine, L-histidine, arginine, glycine, glutamine, low fructans, short chain fatty acids and probiotics.

In another preferred embodiment, the circulating hypovolemic or dehydration symptoms associated with loss of salt are shock and diarrhea.

In a second aspect of the present invention, the present invention provides pyruvate-containing ORS compositions for the uses in the treatment of circulatory hypovolemia and dehydration with salt loss illness, and/or for physiological humoral supplements.

In another preferred embodiment, the present invention provides pyruvate-containing ORS compositions for the uses in the perioperative fluid management, the bowel cleansing preparations before surgery and gastric lavage solutions for poisoning; or preparations of sports drinks or health beverages used in ordinary and hypoxic conditions.

In another preferred embodiment, said conditions include burns, shock, cholera, diarrhea, or gastrointestinal tract inflammation. In another preferred embodiment, the burn is a scald.

Accordingly, the present invention improves the WHO-ORS component in order to achieve the direct protection and preservation of intestinal metabolism and function, which are impaired by hypoxia and/or inflammatory, and further benefits in the clinical outcomes.

### DETAILED DESCRIPTION OF THE INVENTION

The inventors unexpectedly found in the animal studies on the routes of delivering pyruvate that the enteral route to give the pyruvate-containing sugar and salt solution can improve the gut mucosal blood flow, decrease barrier dysfunction and intestinal inflammation, enhance intestinal absorption of water and salt and more effectively correct acidosis, particularly lactic acidosis in animals subjected to burn or scald shock, compared with the original WHO-ORS with bicarbonate or citrate. However, the enteral administration of pure aqueous pyruvate solution without sugar and other salts cannot achieve similar results. The findings suggest that sodium pyruvate contained in the ORS can significantly and directly play a role in the intestinal protection, which WHO-ORS components and the pure pyruvate salt do not. ORS solutions containing sodium pyruvate (Pyr-ORS) retain the easy absorption of Sodium-Glucose and water by intestinal mucosal cells on the basis of merit with WHO-ORS. Further, because of its anti-acid, anti-inflammatory and anti-oxidant effects, pyruvate (sodium pyruvate, calcium pyruvate or a mixture of both) replaced sodium bicarbonate or citrate in WHO-ORS can effectively reduce intestinal ischemia and rehydration (reperfusion) injury, which exists in intestinal tissues due to local acidosis and inflammatory and oxidative damage, improving the intestinal environment for the absorption of water and electrolytes. Therefore, pyruvate-containing compositions of oral rehydration salt, compared with WHO-ORS, can further improve the recovery of hypovolemic shock, and to some extent, can replace intravenous rehydration. Recent animal experiments have confirmed the effects above. On the basis of the above findings, the present invention has been completed.

The pyruvate protection of cells/organs has been widely reported previously. Its improvements of glucose metabolism and cell hypoxia tolerance under hypoxic conditions, direct or indirect antioxidant/nitrosative stress responses, correction of acidosis and other characteristics have also been elucidated. However, the above findings are results from intravenous, intraperitoneal or arterial routes of pyruvate administrations, it has never been reported that oral or enteral pyruvate in a single ingestion or a combination with sugar and salt solution can preserve and enhance the intestinal absorption of water and salts. In humans, except the systemic protection including, heart and liver by systemic administrations of pyruvate, large oral doses of calcium salts: 7-25 g/time or 7 g/day for a week, or even 10 g/day lasted for 30 days did not confirm the improvement of systemic glucose metabolism and organ function, as well as athletic performance or weight loss. Therefore, oral sodium pyruvate has never been routinely used in clinical settings. And the invention for the first time discovered that oral sodium pyruvate-containing sugar and salt solutions, other than a single pyruvate ingestion, can significantly promote systemic and splanchnic blood circulation, correct local intestinal and systemic acidosis, inhibit inflammatory and oxidative damage and improve the intestinal ischemia, intestinal barrier function and water and salt absorption, significantly raising the efficiency of shock recovery in animals.

### Compositions

As used herein, ‘the present invention provides a composition for oral administration of sodium pyvuvate’, ‘the present invention provides oral rehydration salts of sodium pyruvate’ and ‘the present invention provides sodium pyruvate ORS’ means that they contain the following essential components: (i) 2.0-6.0 parts by weight of sodium pyruvate; (ii) 1.5-17.0 parts by weight of sodium chloride; (iii) 0-2.0 parts by weight of potassium chloride or corresponding equivalents of potassium citrate; and (iv) 10.0-50.0 parts by weight of anhydrous glucose or other carbohydrates, but the composition containing sodium bicarbonate and/or sodium citrate basically.

Preferably wherein the amount of sodium pyruvate is 3.0-4.0 parts by weight, optimum amount of 3.5 parts by weight; preferably sodium chloride content is 2.0-5.0 parts by weight, more preferably content of 2.0-3.5 parts by weight, the best content of 2.5-3.5 parts by weight; preferably potassium chloride or an corresponding equivalent of potassium citrate is 1.0-1.8 parts by weight, optimum amount of 1.5 parts by weight; the preferred amount of anhydrous glucose is 13.5-50.0 parts by weight, more preferably an amount of 13.5 to 20.0 parts by weight, or corresponding amounts of other carbohydrates.

The present invention provides sodium pyruvate-containing oral compositions probably in a solid form, such as, but not limited to, powdered; also in a liquid form, in one embodiment, the liquid form of an aqueous solution: per 1000 ml of the solution comprising (a) 2.0-6.0 g of sodium pyruvate; (b) 1.5-17.0 g of sodium chloride; (c) 0-2.0 g of potassium chloride or corresponding equivalents of potassium citrate; and (d) 10.0-50.0 g of anhydrous glucose or corresponding amounts of other carbohydrates; and per 1,000 ml of the solution, preferably comprising sodium pyruvate content of 3.0-4.0 g, optimum content of 3.5 g; preferably comprising sodium chloride content of 2.0-17.0 g, more better content of 2.0-5.0 g, optimum content of 2.5-3.5 g; preferably comprising potassium chloride or corresponding equivalents of potassium citrate content of 1.0-1.8 g, preferred amount of 1.5 g; preferred amount of anhydrous glucose content of 13.5-50.0 g, more preferably an amount of 13.5 to 20.0 g, or appropriate amounts of other carbohydrates.

The present invention provides a composition for oral administration of sodium pyruvate in four essential components. The content of each component can be individually or jointly in their better values, more better values or optimum values.

The present invention provides sodium pyruvate-containing ORS, which can be classified according to their osmolarity: hypertonic oral solutions, isotonic oral solutions and hypotonic oral solutions. The osmolality of hypertonic ORS is 300-1,000 mOsm/L, per 1,000 ml of the oral solution containing hypertonic sodium chloride 3.5-17.0 g and anhydrous glucose 20.0-50.0 g or appropriate amounts of other carbohydrates. The osmolality of hypotonic oral ORS is 120-280 mOsm/L, per 1,000 ml of isotonic (280-300 mOsm/L) or hypotonic oral solution containing sodium chloride is less than 3.5 g and anhydrous glucose less than 20.0 g, or corresponding amounts of other carbohydrates. The present invention provides a composition for oral administration of sodium pyruvate, which should be in a liquid form in the clinical application, a preferred way in practical applications, dissolving the compositions of solid forms in drinking water before use. The mentioned concentration and osmolality above refer to their amounts in dissolved conditions.

If the present invention provides an oral composition containing sodium pyruvate, which is stored in a liquid form, to consider the stability of aqueous pyruvate solutions, the general pH of the solution is 4.0-4.9 adjusted with low concentrations of HCl, or pyruvic acid.

As used herein, “other carbohydrates” includes hydrous glucose, starch and cereals. The cereals include rice, millet, maize, sorghum and wheat, etc. Grains used in the present invention can be powder grain products, such as, but not limited to, rice flour, corn flour, and sorghum flour. In practical applications, they replace or partly replace 20 g of anhydrous glucose and the dosage of rice and others is usually 50-80 grams in the treatment of diarrhea, which may reduce the fluid loss of diarrhea and volume of stools. They may be superior to glucose in this respect.

As used herein, “oral rehydration salts (ORS)” is in a solid form: dusts, powders, granules, foaming agents or tablets. “Oral rehydration solutions (ORS)” refers to solutions of the powders, etc. dissolved in water.

### Uses

The present invention provides hypertonic and isotonic oral rehydration solutions for the treatment of severe burns (including scalds) with or without shock.

The present invention provides isotonic oral rehydration solutions for the treatment of circulatory hypovolemia or dehydration with salt loss induced by a variety of causes, with or without shock.

The present invention provides various oral rehydration solutions for the treatment of diarrhea or cholera.

The present invention provides hypotonic oral rehydration solutions, which are more suitable for children and adults to treat non-cholera diarrhea, acute and chronic inflammation of the gastrointestinal tract associated with loss of salts and water, with or without shock.

The present invention provides hypertonic and isotonic oral rehydration solutions and modified ORS, which are also applicable to the perioperative fluid management, preoperative bowel cleansing preparations in general surgery and obstetric and gynecologic surgery, as well as food or chemical poisoning lavage solutions.

The present invention provides hypotonic ORS solutions, which can be also used as physiological humoral supplements, or modified as beverages and sports drinks, which contain sodium pyruvate and/or calcium pyruvate, sugar and salts, suitable for general or special conditions, such as: athletic performance, Alpine hypoxia and long-term stay in deep-seas.

The present invention provides sodium pyruvate-containing ORS, which is also applicable to various clinical conditions of mammals in addition to human beings.

The above features of the present invention, or the features mentioned in embodiments below can be arbitrarily combined. In this case all of the features revealed in specifications can be used with any form of other compositions; various features disclosed in the specification in the case can be substituted with available alternative features, which have the identical, equal or similar purposes. Therefore, unless otherwise specified, the disclosed features herein are only general examples of equal or similar features.

### The Main Advantages of the Present Invention

1. The present invention provides an oral composition contained sodium pyruvate, which is able to enhance the absorption of ORS (water, electrolytes and glucose), thereby more increasing the circulatory volume, which is superior to the conventional WHO-ORS.
2. The present invention provides an oral composition contained sodium pyruvate, which holds super alkalizing function to correct metabolic acidosis, being more suitable for the correction of severe acidosis, particularly lactic acidosis, complicated with refractory shock and severe diarrhea, compared with the conventional WHO-ORS with bicarbonate or citrate as an alkaline agent.
3. The present invention provides an oral composition contained sodium pyruvate, which has alkalizing, anti-inflammatory and anti-oxidant effects that are basically absent in WHO-ORS, enabling to increase the intestinal blood flow, enhance the tolerance of intestinal epithelial cells to hypoxia, reduce intestinal ischemia and rehydration (reperfusion) injury, which is presented with local intestinal tissue acidosis and inflammatory and oxidative damage, protect the function of intestinal epithelial barrier, improve the intestinal environment for the absorption of electrolytes, sugar and water and then help to correct systemic metabolic disorders and to protect multi-organ function. Thus, pyruvate-containing Pyr-ORS can further promote the recovery of hypovolemic shock, compared with WHO-ORS. To a certain extent, it is more effective alternative than WHO-ORS to intravenous rehydration or reduction of the amount of intravenous fluids.

Next, with combination to specific embodiments, further illustrates the present invention. It should be understood that these embodiments are merely illustrative of the invention and are not intended to limit the scope of the invention. Examples in following specific conditions, where experimental methods are not indicated, usually follow conventional conditions or in accordance with the conditions recommended by the manufacturer. Unless otherwise indicated, all percentages, ratios, proportions, or parts are accounted by weight.

In the present invention, the unit of the weight percentage of volume is well known to the skilled in the art, for example, refers to the solute weight of a solution in 100 ml. Unless otherwise defined, all professional and scientific terms used in the text are identical to those used by the skilled in the art in the familiar sense. In addition, any methods and materials similar or equivalent to the content described with the present methods can be applied. The preferred embodiments in methods and materials described in the text are for demonstration purposes only.

For the prior art WHO-ORS, in the following embodiments, the oral rehydration solution contained pyruvate (Pyr-ORS), obtained by the pyruvate replacement of bicarbonate/citrate in the present invention was investigated with the method described in the literature through a series of experiments.

### Preparation Example 1

### Sodium Pyruvate-Containing Oral Rehydration Solution (Pyr-ORS1)

The substances listed in the following table in accordance with the mixture formulation are dissolved in water to give Pyr-ORS 1:

TABLE 1
Hypertonic sodium pyruvate solution of oral rehydration salts

| Essential component | content (g)/1,000 ml | molecular weight (MW) | osmolality (mOsm/L) |
|---|---|---|---|
| Sodium pyruvate | 3.5 | 110.0 | 64 |
| Sodium chloride | 3.5 | 58.5 | 120 |
| Potassium cloride | 1.5 | 74.5 | 40 |
| Glucose (anhydrous) | 20.0 (2% concentration) | 180.0 | 111 |
| Total osmolarity | | | 335 |

### Preparation Example 2

### Sodium Pyruvate-Containing Oral Rehydration Solution (Pyr-ORS2)

The substances listed in the following table in accordance with the mixture formulation are dissolved in water to give Pyr-ORS2:

TABLE 2
Hypertonic sodium pyruvate solution of oral rehydration salts

| Essential component | content (g)/1,000 ml | molecular weight (MW) | osmolality (mOsm/L) |
|---|---|---|---|
| Sodium pyruvate | 3.5 | 110.0 | 64 |
| Sodium chloride | 17.0 | 58.5 | 581 |
| Potassium cloride | 1.5 | 74.5 | 40 |
| Glucose (anhydrous) | 50.0 (5% concentration) | 180.0 | 278 |
| Total osmolarity | | | 953 |

### Preparation Example 3

### Sodium Pyruvate-Containing Oral Rehydration Solution (Pyr-ORS3)

The substances listed in the following table in accordance with the mixture formulation are dissolved in water to give Pyr-ORS3:

TABLE 3
Isotonic sodium pyruvate solution of oral rehydration salts

| Essential component | content (g)/1,000 ml | molecular weight (MW) | osmolality (mOsm/L) |
|---|---|---|---|
| Sodium pyruvate | 3.5 | 110.0 | 64 |
| Sodium chloride | 3.0 | 58.5 | 102 |
| Potassium cloride | 1.5 | 74.5 | 40 |
| Glucose (anhydrous) | 16.5 (1.65% concentration) | 180.0 | 92 |
| Total osmolarity | | | 298 |

### Preparation Example 4

### Sodium Pyruvate-Containing Oral Rehydration Solution (Pyr-ORS4)

The substances listed in the following table in accordance with the mixture formulation are dissolved in water to give Pyr-ORS4:

TABLE 4
Hypotonic sodium pyruvate solution of oral rehydration salts

| Essential component | content (g)/1,000 ml | molecular weight (MW) | osmolality (mOsm/L) |
|---|---|---|---|
| Sodium pyruvate | 3.5 | 110.0 | 64 |
| Sodium chloride | 2.0 | 58.5 | 68 |
| Potassium cloride | 1.5 | 74.5 | 40 |
| Glucose (anhydrous) | 13.5 (1.35% concentration) | 180.0 | 75 |
| Total osmolarity | | | 247 |

### Experimental Examples

The shock and multi-organ failure laboratory, Burns Institute, First Affiliated Hospital of People's Liberation Army General Hospital, Beijing, has completed the following experiments on Pyr-ORS effects on animals (dogs and rats), in vivo, subjected to shock with burn/scald.

The following experiments used hypertonic Pyr-ORS1 in the example 1 as an example demonstrated that pyruvate-containing compositions in the present invention were superior to conventional compositions in WHO-ORS, confirming that pyruvate replaced bicarbonate or citrate in Pyr-ORS is advantageous over three representative formulations in WHO-ORS with bicarbonate or citrate.

### Determinations

1. Determination of jejunum H₂O and Na⁺ absorption, measured with conventional methods: see ref⁽¹⁵⁻¹⁶⁾;
2. Determination of intestinal mucosal blood flow, measured by using laser Doppler flowmetry (PeriFlux Systm 5000. Sweden RERIMED company): see ref^{(13, 17)};
3. Determination of intestinal mucosa carbon dioxide pressure (PC0₂, using PC0₂ tension tester (Finland, Tonocap type Tonometry), measured with the conventional experimental method modified: see ref⁽¹⁸⁾;

Burn/scald reduces the gastrointestinal blood flow, leading gastrointestinal mucosa to ischemia and hypoxia in animals, the accumulation of acidic metabolites causes an increase of PC0₂, and a decrease of pH value in gastrointestinal mucosa. Studies have shown that the pH values of gastrointestinal mucosa were reduced with the shock aggravation: PC0₂ is negatively correlated with the pH value, reflecting that the PC0₂ is a sensitive indicator of the local mucosal acidosis.

### Experimental Procedures

Materials: obtained the sodium pyruvate-containing oral solution in Preparation Example 1 (Pyr-ORS1, a total osmolarity of 335 mOsm/L).

According to the standard WHO oral rehydration salt (WHO-ORS) formula I (sodium chloride 3.5 g, potassium chloride 1.5 g, sodium bicarbonate 2.5 g and 20.0 g of glucose per liter, total osmolarity of 331 mOsm/L) obtained as a control WHO-ORS1. According to the WHO oral rehydration salt (WHO-ORS) formula II (sodium chloride 3.5 g, potassium chloride 1.5 g, sodium citrate 2.9 g and 20.0 g of glucose per liter, total osmolarity of 311 mOsm/L) obtained as a control WHO-ORS2.

Methods: By gastrointestinal stoma surgery, ORS solutions were infused in 35% total body surface area (TBSA) burned rats (n=10) subjected to hypovolemic shock and the Pyr-ORS1 was compared with the WHO-ORS 1 in respect to the efficacy of shock recovery after injury, the amount of oral solutions infused was according to clinical routine burns rehydration formula: Parkland formula:

the infusion volume=4 ml·kg⁻¹·(1% TBSA)⁻¹ after injury during the first 24 hours.

### Results and Data:

1. Effects of Pyr-ORS on water and Na⁺ absorption rates, intestinal blood flow and mucosal PC0₂ in scald rats

TABLE 5
Intestinal water and [Na⁺] absorption rates, mucosal blood flow and PC0₂ in
rats with 35% TBSA III degree burns infused with ORS 4 hours after injury

| | Scald + | Scald + | Sham + | Sham + |
|---|---|---|---|---|
| n = 10 | Pyr-ORS1 | WHO-ORS1 | Pyr-ORS1 | WHO-ORS1 |
| H₂O absorption | 22.88 ± 1.88*^{#} | 18.14 ± 0.73* | 41.23 ± 1.25 | 40.24 ± 2.07 |
| (mmol · m⁻¹ · h⁻¹) | | | | |
| [Na⁺] absorption | 1.01 ± 0.08*^{#} | 0.85 ± 0.06* | 1.55 ± 0.06 | 1.36 ± 0.05 |
| (mmol · m⁻¹ · h⁻¹) | | | | |
| Mucosal Blood | 114.25 ± 3.62*^{#} | 106.88 ± 5.36* | 149.75 ± 4.40 | 145.00 ± 3.96 |
| Flow (PU) | | | | |
| Mucosal PCO₂ | 45.22 ± 5.08*^{#} | 55.60 ± 6.32* | 36.76 ± 4.23 | 32.6 ± 4.16 |
| (mmHg) | | | | |

Note:
*P < 0.05, vs. corresponding Group Sham;
^{#}P < 0.05, vs. corresponding Group WH0-0RS1.

The experimental results (Table 5) showed that Group Pyr-ORS1 (containing sodium pyruvate) increased 26.1% of intestinal water absorption rate, 18.8% of sodium absorption rate and 6.9% of intestinal blood flow and decreased 18.7% of intestinal PC0₂ 4 hours after injury, compared with Group WHO-ORS1 (containing sodium bicarbonate). The indicators were significantly different between Group Pyr-ORS1 and Group WHO-ORS1 (p<0.05).

Description: Results above demonstrated that the Pyr-ORS1 provided in the present invention was significantly better than the standard WHO-ORS1 in promoting the intestinal water and salt absorption, increasing intestinal mucosal blood flow and correcting local acidosis.

TABLE 6
Intestinal water and [Na⁺] absorption rates and mucosal blood flow in rats
with 35% TBSA III degree burns infused with ORS 4 hours after injury

| | Scald + | Scald + | Sham + | Sham + |
|---|---|---|---|---|
| n = 10 | Pyr-ORS1 | WHO-ORS1 | Pyr-ORS1 | WHO-ORS1 |
| H₂O absorption | 24.87 ± 1.644^{#} | 21.66 ± 1.06 | 41.69 ± 3.25^{Δ} | 36.41 ± 1.85 |
| (mmol · m⁻¹ · h⁻¹) | | | | |
| [Na⁺] absorption | 1.06 ± 0.07^{#} | 0.95 ± 0.05 | 1.54 ± 0.07^{Δ} | 1.43 ± 0.04 |
| (mmol · m⁻¹ · h⁻¹) | | | | |
| Mucosal Blood | 118.25 ± 4.28^{#} | 109.88 ± 4.74 | 149.75 ± 4.40 | 145.84 ± 4.53 |
| Flow (PU) | | | | |

Note:
^{#}P < 0.05, vs. corresponding Group WHO-ORS2;
^{Δ}P < 0.05, vs. corresponding Group WHO-ORS2.

The experimental results (Table 6) showed that Group Pyr-ORS1 (containing sodium pyruvate) increased intestinal absorption rates of water and sodium, as well as intestinal mucosal blood flow 4 hours after burns, compared with Group WHO-ORS2 (containing sodium citrate).

The indicators were significantly different between the two groups (p<0.05).

Description: Results above demonstrated that the Pyr-ORS 1 provided in the present invention was significantly better than the modified WHO-ORS2 in promoting the intestinal absorption of water and salt and increasing intestinal mucosal blood flow. Additional experimental evidence also suggested that the Pyr-ORS 1 was superior to the WHO-ORS2 in the improvement of the intestinal barrier structure and function (dada not shown).
2. Effects of Pyr-ORS on intestinal mucosal barrier function in scald rats

TABLE 7
Indexes of intestinal barrier function in rats with 35% TBSA III
degree burns infused with ORS 4 hours after injury

| | Scald + | Scald + | Scald + | Sham + | Sham + |
|---|---|---|---|---|---|
| n = 10 | Pyr-ORS1 | WHO-ORS1 | NO ORS | Pyr-ORS1 | WHO-ORS1 |
| DAO acitivity | 59.71 ± 11.89*⁺ | 52.70 ± 4.65*^{#} | 40.82 ± 2.43 | 62.13 ± 7.71* | 59.05 ± 4.73* |
| (U/mgprot) | | | | | |
| iFABP | 420.27 ± 40.36*⁺ | 391.4 ± 14.52*^{#} | 358.42 ± 18.99 | 466.27 ± 20.95* | 425.32 ± 32.95* |
| (pg/ml) | | | | | |

Note:
*P < 0.05, vs. NO ORS;
⁺P < 0.05, vs. corresponding Group WHO-ORS1;
^{#}P < 0.05, vs. corresponding Group Sham.

The intestinal diamine oxidase (DAO) activity and the intestinal fatty acid binding protein (iFABP) content are sensitive indicators to reflect the integrity of intestinal barrier function. Methods of diamine oxidase and fatty acid binding protein assay: see references⁽¹⁹⁻²⁰⁾.

Experimental results showed that the DAO activity and the iFABP level in Group Pyr-ORS1 were not only significantly higher than those in Group Scald with no rehydration, but also significantly higher than those in Group WHO-ORS1. The protection of intestinal barrier function was better by Pyr-ORS1 than by WHO-ORS1 (p<0.05), and the levels of barrier function in Group Pyr-ORS1 were close to the levels in Group Sham (p>0.05). The results indicated that the enteral infusion of Pyr-ORS reduced the damage of intestinal epithelial barrier after scald.

Results in the above two aspects: water and salt absorption and barrier function strongly demonstrate that Pyr-ORS protects intestinal barrier function after burn injury and promotes the absorption of water and salt in the impaired intestine, playing more beneficial roles in shock resuscitation, compared with WHO-ORS.
3. Effects of Pyr-ORS on the intestinal Na⁺-K⁺-ATP enzyme activity and epithelial expression of aquaporin in scald rats

One of important prerequisites to maintain the intestinal barrier integrity is sufficient energy metabolism and ATP supply. Experimental results also showed that burns significantly decreased the intestinal Na⁺-K⁺-ATP activity, but the activity was maintained in a significantly higher level in Group Pyr-ORS1 than in Group WHO-ORS1 (p<0.05) (data not shown). More studies with the intestinal immunohistochemical assay showed that the expression of intestinal epithelial aquaporins (Aquaporin-1, AQP1) in the Pyr-ORS1 group was significantly preserved, which was apparently superior to that in the WHO-ORS1 group. Further observations also indicated that the systemic disturbance in both glucose metabolism and acid-base balance was significantly improved with Pyr-ORS1 than with WHO-ORS1 (data not shown).

All the above indicate that the enteral Pyr-ORS significantly protects intestinal function and enhances the absorption of water and salts, as well as improves the systemic metabolism in rats.

4. Effects of Pyr-ORS on enteral resuscitation of hypovolemic shock induced by 50% TBSA III degree burns in Beagle dogs models

The model of hypovolemic shock induced by 50% TBSA III degree burns in Beagle dogs was developed as described in the reference⁽²¹⁾.

Beagle dogs subjected to burn shock were divided into three groups: no rehydration group (n=8), oral pyruvate (Pyr-ORS1) group (n=10) and oral bicarbonate (WHO-ORS1) group (n=10). The ORS was intra-gastrically infused immediately after injury, according to clinical routine burns rehydration formula (parkland formula: 4 ml·kg⁻¹·% TBSA⁻¹) calculated in the first 24 hours with Pyr-ORS1 and WHO-ORS1, respectively, compared with the no rehydration. The mean arterial pressure and blood volume in the Pyr-ORS1 group were significantly increased and its 24-hour survival rate was 60%, which was significantly higher than those in the no rehydration group (0%) and the WHO-ORS1 group (40%). The indicators of heart, liver and intestinal function 6 and 24 hours after injury in the Pyr-ORS1 group were better than those in the WHO-ORS1 group. Results with fluorescent dextran labeling also demonstrated that the microvascular permeability of organs 6 hours after burns in the former was significantly lower than those in both the no rehydration and WHO-ORS1 groups (data not shown).

The above experimental results in rats and dogs, in vivo, subjected to burn/scald injury and intragastrically rehydrated with Pyr-ORS strongly demonstrate the effectiveness and superiority of sodium pyruvate to replace sodium bicarbonate/sodium citrate in WHO-ORS in the increase of intestinal absorption of water and salts, correction of hypovolemia, improvement of local and systemic organ metabolism and function and the survival in the present invention.

All the literatures mentioned in the present invention are applied as references, as if they were individually cited in articles as a reference.

It should be also understood that the skilled in the art after reading the all content of the present invention can make various modifications to the present invention in equivalent forms, which are all covered in the present applications as defined by the appended claims scope.

### REFERENCES

1. Hu S, et al: Oral rehydration-use of fluid resuscitation of burn shock in war, sudden accidents or disasters. Med J Chin PLA 2008; 33 (6): 635-6 (in Chinese).
2. Hu S, et al: Oral or enteral fluid resuscitation of burn shock. Chin J Surg 2009; 47(24):1638-40 (in Chinese).
3. The Lancet: Water with sugar and salt. Lancet. 1978; 2(8084): 300-1.
4. Santosham M, et al: Progress and barriers for the control of diarrhoeal disease. Lancet 2010; 376(9734):63-7.
5. Michell MW, et al: Enteral resuscitation of burn shock using World Health Organization oral rehydration solution: a potential solution for mass casualty care. J Burn Care Res 2006; 27(6):819-25.
6. Venter M, et al: Enteral resuscitation and early enteral feeding in children with major burns—effect on McFarlane response to stress. Burns 2007; 33(4):464-71.
7. Hu S, et al: Effects of early oral fluid resuscitation on organ functions and survival during shock stage in dogs with a 50% total body surface area full-thickness burn. Nat Med J China 2008; 88 (44): 3149-52 (in Chinese).
8. Hu S, et al: Effects of oral rehydration on hemodynamics and microcirculatory perfusion in dogs with fatal hemorrhagic shock. Chin J Anesthesiol 2010; 30 (4): 448-51 (in Chinese).
9. Hu S, et al: Effects of early oral fluid resuscitation on hemodynamic and tissue perfusion during shock stage in dogs with a 50% total body surface area full-thickness burn. Chin J Surg 2009; 47 (19): 1499-1502 (in Chinese).
10. Hassoun H T, et al: Post-injury multiple organ failure: the role of the gut. Shock 2001; 15(1):1-10.
11. Pulungsih SP, et al: Standard WHO-ORS versus reduced-osmolarity ORS in the management of cholera patients. J Health Popul Nutr 2006; 24(1):107-12.
12. Hu S, et al: Carbachol promotes gastrointestinal function during oral resuscitation of burn shock. World J Gastroenterol 2011; 17(13): 1746-52.
13. Bao C, et al: Effect of carbachol on intestinal mucosal blood flow, activity of Na⁺-K⁺-ATPase, expression of aquaporin-1, and intestinal absorption rate during enteral resuscitation of burn shock in rats. J Burn Care Res 2010; 31(1):200-6.
14. Alam N H, et al: L-isoleucine-supplemented oral rehydration solution in the treatment of acute diarrhoea in children: a randomized controlled trial. J Health Popul Nutr 2011; 29(3):183-90.
15. Cooper H, et al: A method for studying absorption of water and solute from the human small intestine. Gastroenterology 1966; 50(1):1-7.
16. Hu S, et al: Efficiency of intestinal absorption to glucose electrolyte solution during enteral rehydration in 35% TBSA scalded rats. Med J Chin PLA 2008; 33 (6): 640-2 (in Chinese).
17. Geng S J, et al: Effects of carbachol on intestinal mucosal blood flow, activity of Na+-K+-ATP and expression of aquaporin-1 during the enteral rehydration of scald rats. Med J Chin PLA 2008; 33 (6): 649-51 (in Chinese).
18. Kozar R A, et al: Specific intraluminal nutrients alter mucosal blood flow during gut ischemia/reperfusion. JPEN J Parenter Enteral Nutr 2002; 26(4):226-9.
19. Li J Y, et al: Determinations of the diamine oxidase activity in blood and intestinal tissues by spectrophotometry. Amino acids and biological resources 1996; 18 (1): 28-30 (in Chinese).
20. Guo Q H, et al: Advances in the immunoassay of fatty acid-binding protein. Chin J Geront 2007; 27(24):70-2 (in Chinese).
21. Che J W, et al: Development of animal model of early oral fluid resuscitation of burn shock. Chin J Trauma 2009; 25 (3): 259-63 (in Chinese).

1. An oral rehydration salt composition contains sodium pyruvate (Pyruvate) for the treatment of circulatory hypovolemia or dehydration with salt loss, the composition comprising:
(i) 2.0-6.0 parts by weight of sodium pyruvate;
(ii) 1.5-17.0 parts by weight of sodium chloride;
(iii) 0-2.0 parts by weight of potassium chloride or corresponding equivalents of potassium citrate; and
(iv) 10.0-50.0 parts by weight of anhydrous glucose or other carbohydrates, wherein the components (i)+(ii)+(iii)+(iV) by weight constitutes 50-100% of the total weight of composition.

2. The composition according to claim 1, wherein said composition comprises:
(i) 3.0-4.0 parts by weight of sodium pyruvate;
(ii) 2.0-5.0 parts by weight of sodium chloride;
(iii) 1.0-1.8 parts by weight of potassium chloride or corresponding equivalents of potassium citrate; and
(iv) 13.5-20.0 parts by weight of anhydrous glucose or other carbohydrates.

3. The composition according to claim 1, wherein said composition is an oral solution containing in 1,000 ml:
(a) 2.0-6.0 g of sodium pyruvate;
(b) 1.5-17.0 g of sodium chloride;
(c) 0-2.0 g of potassium chloride or corresponding equivalents of potassium citrate; and
(d) 10.0-50.0 g of anhydrous glucose or other carbohydrates.

4. The composition according to claim 3, wherein said solution has a composition in 1,000 ml containing:
(a) 3.0-4.0 g of sodium pyruvate;
(b) 2.0-5.0 g of sodium chloride;
(c) 1.0-1.8 g of potassium chloride or corresponding equivalents of potassium citrate; and
(d) 13.5-20.0 g of anhydrous glucose or other carbohydrates.

5. The composition according to claim 4, wherein
osmotic pressure of the solution is 300-1,000 mOsm/L, and
the solution containing in 1,000 ml not less than 3.5 g of sodium chloride and containing not less than 20.0 g of anhydrous glucose or other carbohydrates.

6. The composition according to claim 4, wherein
osmotic pressure of the solution is 120-280 m0sm/L, and
the solution containing in 1,000 ml less than 3.5 g of sodium chloride and containing less than 20.0 g of glucose or other carbohydrates.

7. The composition according to claim 1, wherein said composition further contains zinc, magnesium, selenium and calcium salts, phosphates, antioxidants, gastrointestinal motility vasoactive components, immune nutrients, herbal ingredients and/or flavoring agents.

8. The composition according to claim 7, wherein
said antioxidants are selected from the following combination of one or more of: vitamin C, vitamin E, N-acetylcysteine, and Pentoxifylline;
gastrointestinal motility and the vasoactive components are selected from the following combination of one or more of: Carbachol, Mosapride, Anisodamine, and Dobutamine; and
immune nutrients are selected from the following combination of one or more of: L-isoleucine, L-histidine, arginine, glycine, glutamine, fructooligosaccharides (low fructans), short chain fatty acids, and probiotics.

9. The composition according to claim 1, wherein said composition is used in conditions to treat circulatory hypovolemia and dehydration with salt loss disorders and/or for physiological humoral supplements.

10. The composition according to claim 9, wherein said conditions for the treatments include burn (scald), shock, cholera, diarrhea, gastrointestinal tract inflammation, or perioperative fluid managements.
